(19) United States
(12) Patent Application Publication
(10) Pub. No.: US 2023/0143386 A1
(43) Pub. Date: May 11, 2023

(54) ORAL DRUG TABLET MANUFACTURING DEVICE AND MANUFACTURING METHOD THEREOF

(71) Applicants: YEN-SHU LIN, TAIPEI (TW); CHIH-CHIA TSAI, TAIPEI (TW)

(72) Inventors: YEN-SHU LIN, TAIPEI (TW); CHIH-CHIA TSAI, TAIPEI (TW)

(73) Assignee: Taiwan Mercury Medical Corporation, TAIPEI (TW)

(21) Appl. No.: 17/520,516

(22) Filed: Nov. 5, 2021

(51) Int. Cl. A61K 9/20; A61K 9/00; A61J 3/10; B30B 11/00

(52) U.S. Cl. CPC A61K 9/2095; A61K 9/0053; A61J 3/10; B30B 11/006

(57) ABSTRACT

An oral drug tablet manufacturing device and a manufacturing method thereof comprise a tablet manufacturing area, a micro-control unit, and a collecting area. The tablet manufacturing area comprises a tablet spraying device having a roller and a piezoelectric nozzle, a medicine powder filling container is communicated to the piezoelectric nozzle, the medicine powder filling container contains a plurality of medicine powders, the medicine powders are sprayed and printed through the piezoelectric nozzle, and then the medicine powders are pressed and flattened through the roller to cause the medicine powders to form a drug tablet. The micro-control unit is disposed in the piezoelectric nozzle, the micro-control unit has a storage module and an execution control module. The storage module stores a tablet spray information, the execution control module controls the tablet spraying device through the tablet spray information, and the collecting area is used to collect the drug tablet.

### BACKGROUND OF THE INVENTION

### Field of Invention

The invention relates to an oral drug tablet manufacturing device, and more particularly to an oral drug tablet manufacturing device and a manufacturing method thereof capable of effectively controlling a quantity of layers of medicine powder and colloid to reduce a thickness of a drug tablet.

### Related Art

Drug tablet on the market is one of the most common types of drug bags, the current method for manufacturing drug tablet is to slide the mixed medicine powder into the tablet press machine from the upper funnel, the mixed medicine powder enters the powder tank and falls into a plurality of sets of tablet forming molds, the size of the space in the mold determines the volume and thickness of the tablet. After entering the mold, the medicine powder will first have a preliminary appearance according to the shape of the mold, the structure is loose, and the thickness is about 2-3 times of that of the final product, then the upper and lower molds are pressed against each other to press the loose medicine powder into a solid tablet that can withstand collisions. Finally, the lower mold pushes the drug tablet out of the mold upward. By designing the shape of the mold to determine the appearance and size of the drug tablet, and then through the manufacturing process of filling, extrusion, and mold rotation by machines, tens of thousands of the same tablet can be manufactured in a short time. This conventional method for making drug tablet requires not only mixing the ingredients and proportions of the medicines, but also requires forming the drug tablet in a mold. Therefore, every type of drug tablet requires a compression molding tool. Due to the problem of wear and tear of compression molding tool, it is necessary to prepare multiple sets of the same compression molding tool, which leads to high overall costs. When the production volume of a special drug tablet is not high, the unit price of the drug tablet cannot be reduced. Moreover, the volume formed by the thickness and area in the conventional drug tablet manufacturing method is limited by the mold, and the volume of the drug tablet cannot be adjusted whenever necessary according to requirements.

Therefore, the inventor of the invention and relevant manufacturers engaged in this industry are eager to research and make improvement to solve the above-mentioned problems and drawbacks in the prior art.

### SUMMARY OF THE INVENTION

Therefore, in order to effectively solve the above-mentioned problems, a main object of the invention is to provide an oral drug tablet manufacturing device capable of effectively controlling a quantity of layers of medicine powder and colloid to reduce a thickness of a drug tablet.

A secondary object of the invention is to provide an oral drug tablet manufacturing device capable of manufacturing fast and reducing the cost of mold development and production.

Another secondary object of the invention is to provide an oral drug tablet manufacturing method capable of effectively controlling a quantity of layers of medicine powder and colloid to reduce a thickness of a drug tablet.

Yet another secondary object of the invention is to provide an oral drug tablet manufacturing method capable of manufacturing fast and reducing the cost of mold development and production.

In order to achieve the above objects, the invention provides an oral drug tablet manufacturing device comprising a tablet manufacturing area, a micro-control unit, and a collecting area. The tablet manufacturing area comprises a tablet spraying device, the tablet spraying device has a roller and a piezoelectric nozzle, a medicine powder filling container is communicated to the piezoelectric nozzle, the medicine powder filling container contains a plurality of medicine powders, the medicine powders are sprayed and printed through the piezoelectric nozzle, and then the medicine powders are pressed and flattened through the roller to cause the medicine powders to form a drug tablet. The micro-control unit is disposed in the piezoelectric nozzle, the micro-control unit has a storage module and an execution control module. The storage module stores a tablet spray information, the execution control module controls the tablet spraying device through the tablet spray information, and the collecting area is used to collect the drug tablet.

In one embodiment, the tablet manufacturing area and the collecting area are located in a sealed cavity.

In one embodiment, further comprising a colloid filling container communicated to the piezoelectric nozzle, and the colloid filling container containing a colloid and performing a spraying and printing operation through the piezoelectric nozzle.

In one embodiment, further comprising a transition area adjacent to the collecting area, and the drug tablet being transferred from the collecting area to the transition area via opening of a one-way valve.

In one embodiment, the tablet spray information comprises medicine powder ingredients, a quantity of layers of medicine powder, colloid ingredients, a quantity of layers of colloid, and a drug tablet area.

In order to achieve the above objects, the invention provides an oral drug tablet manufacturing method using the oral drug tablet manufacturing device in the previous embodiment, characterized in that, comprising following steps of:

S1: providing a tablet spraying device with a roller and a piezoelectric nozzle;

S2: filling a plurality of medicine powders and a colloid into a medicine powder filling container and a colloid filling container respectively, and communicating the medicine powder filling container and the colloid filling container with the piezoelectric nozzle;

S3: providing a micro-control unit with a storage module and an execution control module, the execution control module controlling the tablet spraying device through a tablet spray information in the storage module for spraying and printing the medicine powders and the colloid through the piezoelectric nozzle, and then pressing and flattening the medicine powders and the colloid by the roller; and

S4: repeatedly spraying, printing, pressing and flattening the medicine powders and the colloid to form a drug tablet.

In one embodiment, the medicine powders at least comprise chloral hydrate, benzodiazepines, zopiclone, zolpidem, and an excipient.

In one embodiment, the medicine powders at least comprise phenytoin, carbamazepine, lamotrigine, valproic acid, clonazepam, oxcarbazepine, Lamictal, Topamax, Neurontin, Sabril, and an excipient.

In one embodiment, the medicine powders at least comprise sildenafil, tadalafil, microcrystalline cellulose, croscarmellose sodium, nitroglycerin, and an excipient.

In one embodiment, the excipient can be microcrystalline cellulose, calcium phosphate polyvinylpyrrolidone, guar gum, sodium stearyl fumarate, xylitol, sodium gluconate, magnesium aluminate, maltose, mannitol, polyvinyl acetate, or sorbitol, or a combination of the above.

Through the design of the invention, the piezoelectric nozzle structure of the tablet spraying device is capable of effectively controlling a quantity of layers of the medicine powders and the colloid to reduce a thickness of the drug tablet, and at the same time achieving fast manufacturing and reducing the cost of mold development and production.

### BRIEF DESCRIPTION OF THE DRAWINGS

FIG. 1 is a perspective view of a first embodiment of an oral drug tablet manufacturing device of the invention;

FIG. 2 is a perspective view of the first embodiment of the oral drug tablet manufacturing device of the invention viewed from another angle;

FIG. 3A is a block diagram of the first embodiment of the oral drug tablet manufacturing device of the invention;

FIG. 3B is a block diagram of a piezoelectric nozzle of the invention;

FIG. 3C is a schematic diagram of the piezoelectric nozzle of the invention;

FIG. 4 is a block diagram of a second embodiment of the oral drug tablet manufacturing device of the invention; and

FIG. 5 is a flow chart of steps of an oral drug tablet manufacturing method of the invention.

### DETAILED DESCRIPTION OF THE INVENTION

The above objects of the invention, as well as its structural and functional features, will be described in accordance with the preferred embodiments of the accompanying drawings.

In the following, for the formation and technical content related to an oral drug tablet manufacturing device and a manufacturing method thereof in the invention, various applicable examples are exemplified and explained in detail with reference to the accompanying drawings; however, the invention is of course not limited to the enumerated embodiments, drawings, or detailed descriptions.

Furthermore, those who are familiar with this technology should also understand that the enumerated embodiments and accompanying drawings are only for reference and explanation, and are not used to limit the invention; other modifications or alterations that can be easily implemented based on the detailed descriptions of the invention are also deemed to be within the scope without departing from the spirit or intention thereof as defined by the appended claims and their legal equivalents.

And, the directional terms mentioned in the following embodiments, for example: “above”, “below”, “left”, “right”, “front”, “rear”, etc., are only directions referring in the accompanying drawings. Therefore, the directional terms are used to illustrate rather than limit the invention. In addition, in the following embodiments, the same or similar elements will be labeled with the same or similar numerals.

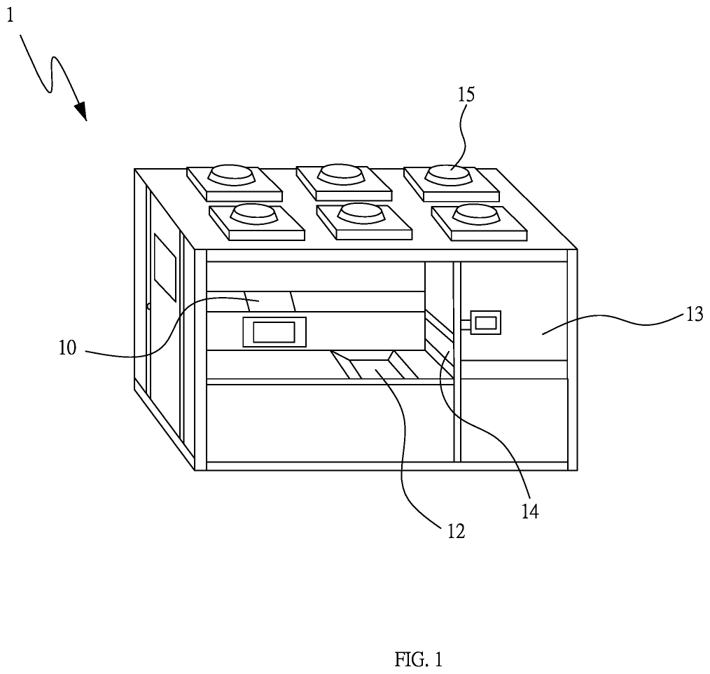

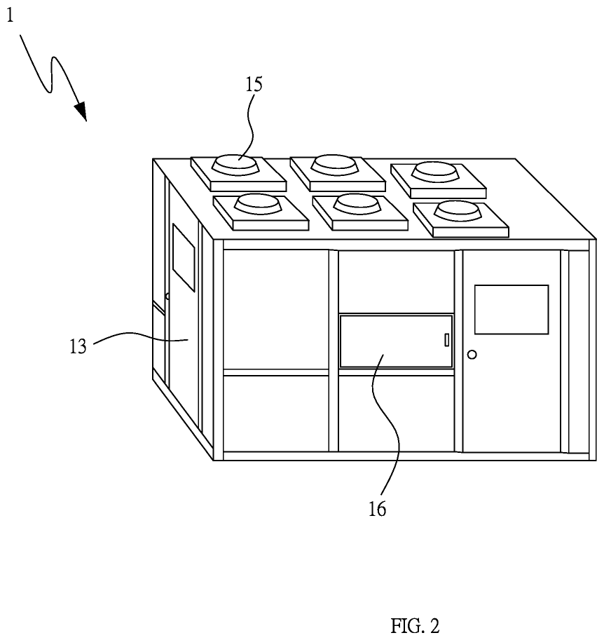

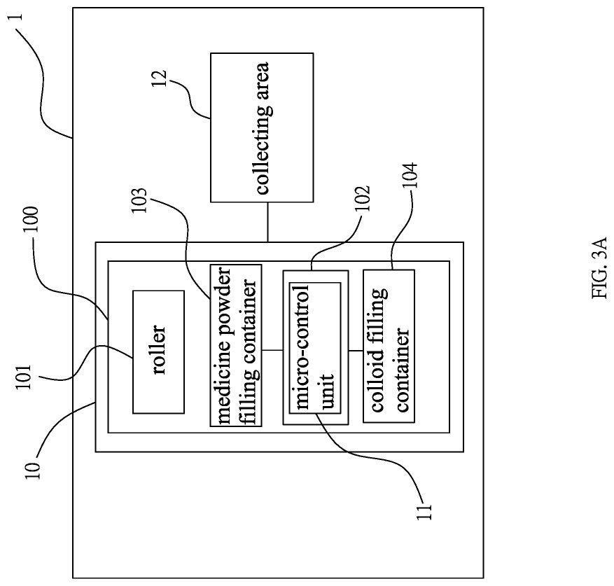

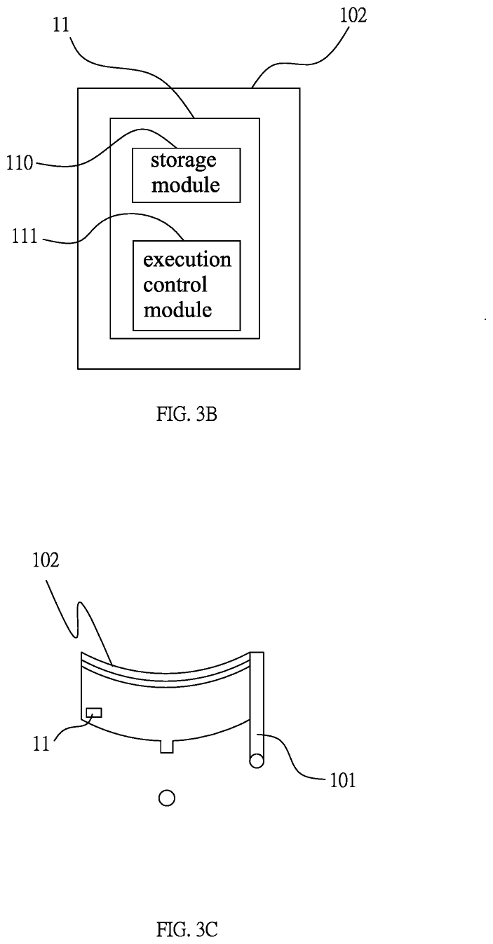

Please refer to FIGS. 1, 2, 3A, 3B, and 3C respectively for a perspective view of a first embodiment of an oral drug tablet manufacturing device of the invention; a perspective view of the first embodiment of the oral drug tablet manufacturing device of the invention viewed from another angle; a block diagram of the first embodiment of the oral drug tablet manufacturing device of the invention; a block diagram of a piezoelectric nozzle of the invention; and a schematic diagram of the piezoelectric nozzle of the invention. As shown in the figures, an oral drug tablet manufacturing device 1 comprises a tablet manufacturing area 10, a micro-control unit 11, and a collecting area 12. The tablet manufacturing area 10 comprises a tablet spraying device 100, the tablet spraying device 100 has a roller 101 and a piezoelectric nozzle 102, a medicine powder filling container 103 is communicated to the piezoelectric nozzle 102, the medicine powder filling container 103 contains a plurality of medicine powders, and the medicine powders are sprayed and printed through the piezoelectric nozzle 102, and then the medicine powders are pressed and flattened through the roller 101, so that the medicine powders form a drug tablet. In addition, the tablet spraying device 100 further has a colloid filling container 104, the colloid filling container 104 is also communicated to the piezoelectric nozzle 102, the colloid filling container 104 contains a colloid and performs a spraying and printing operation through the piezoelectric nozzle 102. In other words, the piezoelectric nozzle 102 sprays the medicine powders and the colloid alternately during operation, so that the laminated or stacked medicine powders and colloid form the drug tablet.

The micro-control unit 11 is disposed in the piezoelectric nozzle 102. The micro-control unit 11 has a storage module 110 and an execution control module 111. The storage module 110 is used to store a tablet spray information. The execution control module 111 controls the tablet spraying device 100 through the tablet spray information. It should be noted that, wherein the tablet spray information comprises medicine powder ingredients, a quantity of layers of medicine powder, colloid ingredients, a quantity of layers of colloid, and a drug tablet area.

The collecting area 12 is adjacent to the tablet manufacturing area 10. The collecting area 12 is used to collect the drug tablet. The tablet manufacturing area 10 and the collecting area 12 are located in a sealed cavity to prevent the tablet manufacturing area 10 and the collecting area 12 from being polluted externally.

It should be particularly noted that the oral drug tablet manufacturing device 1 first fills the medicine powders into the medicine powder filling container 103 when the drug tablet is manufactured. Wherein if the drug tablet is a sleeping pill, the medicine powder ingredients at least comprise chloral hydrate, benzodiazepines, zopiclone, zolpidem, and an excipient; if the drug tablet is an antiepileptic drug, the medicine powder ingredients at least comprise phenytoin, carbamazepine, lamotrigine, valproic acid, clonazepam, oxcarbazepine, Lamictal, Topamax, Neurontin, Sabril, and an excipient; if the drug tablet is a cardiovascular drug, the medicine powder ingredients at least comprise sildenafil, tadalafil, microcrystalline cellulose, croscarmellose sodium, nitroglycerin, and an excipient; and wherein the excipient can be microcrystalline cellulose, calcium phosphate polyvinylpyrrolidone, guar gum, sodium stearyl fumarate, xylitol, sodium gluconate, magnesium aluminate, maltose, mannitol, polyvinyl acetate, or sorbitol, or a combination of the above, in addition, the colloid is filled in the colloid filling container 104.

In addition, it should be noted that the oral drug tablet manufacturing device 1 of the invention further has a fan filter module 15, which is used to form a negative pressure inside the tablet manufacturing area 10, a material reduction area and a transition area 13, and blow off and clean dust inside the tablet manufacturing area 10, the material reduction area and the transition area 13. In addition, the invention further has a central control unit 16, the central control unit 16 is used to control an overall operation of the oral drug tablet manufacturing device 1.

Therefore, after the medicine powder filling container 103 is filled with the medicine powders and the colloid filling container 104 is filled with the colloid, the tablet spraying device 100 can be controlled by the micro-control unit 11 to spray the medicine powders, and the execution control module 111 controls the tablet spraying device 100 through the tablet spray information. Therefore, the execution control module 111 not only controls a spraying amount of the medicine powders, but also controls a spraying amount of the colloid at the same time to spray the medicine powders and the colloid repeatedly and alternately so that the laminated medicine powders and colloid form the drug tablet to complete a prescribed quantity of layers and thickness of the drug tablet on the tablet manufacturing area 10, thereby effectively controlling a quantity of layers of the medicine powders and the colloid to reduce a thickness of the drug tablet, and at the same time achieving fast manufacturing and reducing the cost of mold development and production.

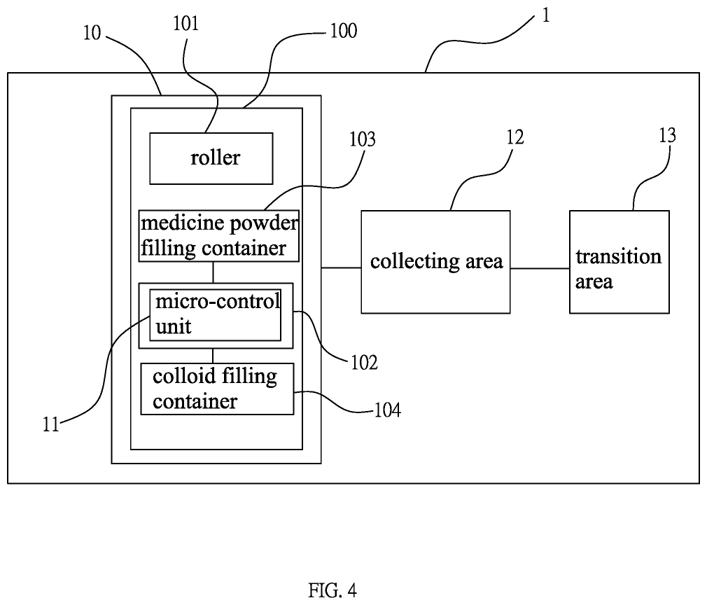

Please refer to FIG. 4 for a block diagram of a second embodiment of the oral drug tablet manufacturing device of the invention. The components and the corresponding relationship between the components of the oral drug tablet manufacturing device 1 in this embodiment are the same as that of the aforementioned oral drug tablet manufacturing device 1, and thus will not be repeated here. However, a main difference between the oral drug tablet manufacturing device 1 and the aforementioned oral drug tablet manufacturing device 1 is that the oral drug tablet manufacturing device 1 further has the transition area 13, the transition area 13 is adjacent to the collecting area 12, a one-way valve 14 is disposed between the transition area 13 and the material reduction area, and the drug tablet is transferred from the collecting area 12 to the transition area 13 via opening of the one-way valve 14 to complete collection operation of the drug tablet.

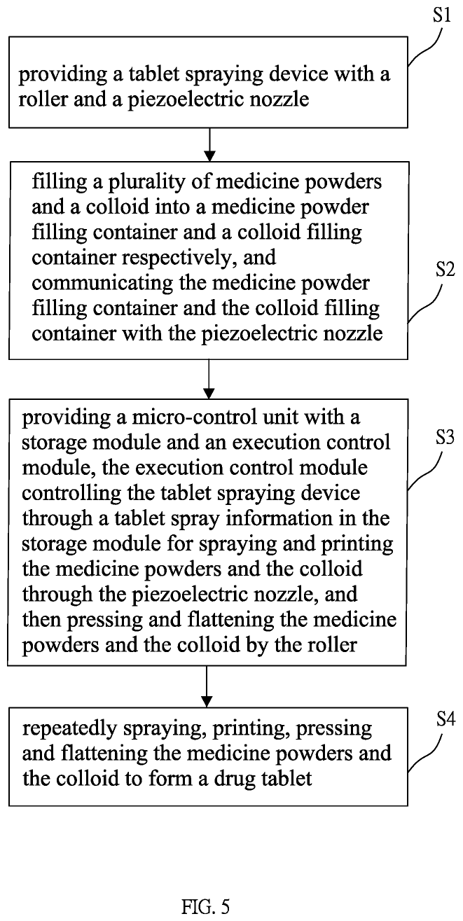

Please refer to FIG. 5 for a flow chart of steps of an oral drug tablet manufacturing method of the invention. As shown in the figure, an oral drug tablet manufacturing method comprising steps of:

S1: providing a tablet spraying device 100 with a roller and a piezoelectric nozzle;

first, providing a tablet spraying device 100, the tablet spraying device 100 being provided with a roller 101 and a piezoelectric nozzle 102 therein;

S2: filling a plurality of medicine powders and a colloid into a medicine powder filling container and a colloid filling container respectively, and communicating the medicine powder filling container and the colloid filling container with the piezoelectric nozzle;

providing a medicine powder filling container 103 and a colloid filling container 104, the medicine powder filling container 103 and the colloid filling container 104 being communicated to the piezoelectric nozzle 102 of the tablet spraying device 100, the medicine powder filling container 103 containing the medicine powders, the colloid filling container 104 containing the colloid;

S3: providing a micro-control unit with a storage module and an execution control module, the execution control module controlling the tablet spraying device through a tablet spray information in the storage module for spraying and printing the medicine powders and the colloid through the piezoelectric nozzle, and then pressing and flattening the medicine powders and the colloid by the roller;

the piezoelectric nozzle 102 being provided with a micro-control unit 11 therein, the micro-control unit 11 having a storage module 110 and an execution control module 111, controlling the tablet spraying device 100 to spray the medicine powders through the micro-control unit 11, the execution control module 111 controlling the tablet spraying device 100 through the tablet spray information, the execution control module 111 not only controlling a spraying amount of the medicine powders, but also controlling a spraying amount of the colloid at the same time for spraying and printing the medicine powders and the colloid through the piezoelectric nozzle 102, and then pressing and flattening the medicine powders and the colloid by the roller 101; and

S4: repeatedly spraying, printing, pressing and flattening the medicine powders and the colloid to form a drug tablet.

Spraying the medicine powders and the colloid repeatedly and alternately so that the laminated medicine powders and colloid forming the drug tablet to complete a prescribed quantity of layers and thickness of the drug tablet on the tablet manufacturing area 10.

Thereby the oral drug tablet manufacturing method of the invention is capable of effectively controlling a quantity of layers of the medicine powders and the colloid to reduce a thickness of the drug tablet, and at the same time achieving fast manufacturing and reducing the cost of mold development and production.

In summary, the invention has the following advantages over the prior art:

1. capable of effectively controlling a quantity of layers of the medicine powders and the colloid to reduce a thickness of the drug tablet; and

2. achieving fast manufacturing and reducing the cost of mold development and production.

The basic principles, main features and advantages of the invention have been shown and described above. Those skilled in the art should understand that the invention is not limited by the above-mentioned embodiments. Descriptions in the above-mentioned embodiments and specification merely illustrate the principles of the invention, without departing from the spirit and scope of the invention, the invention will have various changes and improvements, all these changes and improvements shall fall within the scope of protection of the invention, and the scope of protection of the invention is defined by the appended claims and their equivalents.

What is claimed is:

1. An oral drug tablet manufacturing device comprising:
a tablet manufacturing area comprising a tablet spraying device, the tablet spraying device having a roller and a piezoelectric nozzle, a medicine powder filling container being communicated to the piezoelectric nozzle, the medicine powder filling container containing a plurality of medicine powders, the medicine powders being sprayed and printed through the piezoelectric nozzle, and then the medicine powders being pressed and flattened through the roller to cause the medicine powders to form a drug tablet;
a micro-control unit disposed in the piezoelectric nozzle, the micro-control unit having a storage module and an execution control module, the storage module storing a tablet spray information, the execution control module controlling the tablet spraying device through the tablet spray information; and
a collecting area adjacent to the tablet manufacturing area, the collecting area being used to collect the drug tablet.

2. The oral drug tablet manufacturing device as claimed in claim 1, wherein the tablet manufacturing area and the collecting area are located in a sealed cavity.

3. The oral drug tablet manufacturing device as claimed in claim 1, further comprising a colloid filling container communicated to the piezoelectric nozzle, and the colloid filling container containing a colloid and performing a spraying and printing operation through the piezoelectric nozzle.

4. The oral drug tablet manufacturing device as claimed in claim 1, further comprising a transition area adjacent to the collecting area, and the drug tablet being transferred from the collecting area to the transition area via opening of a one-way valve.

5. The oral drug tablet manufacturing device as claimed in claim 1, wherein the tablet spray information comprises medicine powder ingredients, a quantity of layers of medicine powder, colloid ingredients, a quantity of layers of colloid, and a drug tablet area.

6. An oral drug tablet manufacturing method using the oral drug tablet manufacturing device as claimed in claim 1, characterized in that, comprising following steps of:
S1: providing a tablet spraying device with a roller and a piezoelectric nozzle;
S2: filling a plurality of medicine powders and a colloid into a medicine powder filling container and a colloid filling container respectively, and communicating the medicine powder filling container and the colloid filling container with the piezoelectric nozzle;
S3: providing a micro-control unit with a storage module and an execution control module, the execution control module controlling the tablet spraying device through a tablet spray information in the storage module for spraying and printing the medicine powders and the colloid through the piezoelectric nozzle, and then pressing and flattening the medicine powders and the colloid by the roller; and
S4: repeatedly spraying, printing, pressing and flattening the medicine powders and the colloid to form a drug tablet.

7. An oral drug tablet manufacturing method using the oral drug tablet manufacturing device as claimed in claim 2, characterized in that, comprising following steps of:
S1: providing a tablet spraying device with a roller and a piezoelectric nozzle;
S2: filling a plurality of medicine powders and a colloid into a medicine powder filling container and a colloid filling container respectively, and communicating the medicine powder filling container and the colloid filling container with the piezoelectric nozzle;
S3: providing a micro-control unit with a storage module and an execution control module, the execution control module controlling the tablet spraying device through a tablet spray information in the storage module for spraying and printing the medicine powders and the colloid through the piezoelectric nozzle, and then pressing and flattening the medicine powders and the colloid by the roller; and
S4: repeatedly spraying, printing, pressing and flattening the medicine powders and the colloid to form a drug tablet.

8. An oral drug tablet manufacturing method using the oral drug tablet manufacturing device as claimed in claim 3, characterized in that, comprising following steps of:
S1: providing a tablet spraying device with a roller and a piezoelectric nozzle;
S2: filling a plurality of medicine powders and a colloid into a medicine powder filling container and a colloid filling container respectively, and communicating the medicine powder filling container and the colloid filling container with the piezoelectric nozzle;
S3: providing a micro-control unit with a storage module and an execution control module, the execution control module controlling the tablet spraying device through a tablet spray information in the storage module for spraying and printing the medicine powders and the colloid through the piezoelectric nozzle, and then pressing and flattening the medicine powders and the colloid by the roller; and
S4: repeatedly spraying, printing, pressing and flattening the medicine powders and the colloid to form a drug tablet.

9. An oral drug tablet manufacturing method using the oral drug tablet manufacturing device as claimed in claim 4, characterized in that, comprising following steps of:
S1: providing a tablet spraying device with a roller and a piezoelectric nozzle;
S2: filling a plurality of medicine powders and a colloid into a medicine powder filling container and a colloid filling container respectively, and communicating the medicine powder filling container and the colloid filling container with the piezoelectric nozzle;
S3: providing a micro-control unit with a storage module and an execution control module, the execution control module controlling the tablet spraying device through a tablet spray information in the storage module for spraying and printing the medicine powders and the colloid through the piezoelectric nozzle, and then pressing and flattening the medicine powders and the colloid by the roller; and
S4: repeatedly spraying, printing, pressing and flattening the medicine powders and the colloid to form a drug tablet.

10. The oral drug tablet manufacturing method as claimed in claim 6, wherein the medicine powders at least comprise chloral hydrate, benzodiazepines, zopiclone, zolpidem, and an excipient.

11. The oral drug tablet manufacturing method as claimed in claim 6, wherein the medicine powders at least comprise phenytoin, carbamazepine, lamotrigine, valproic acid, clonazepam, oxcarbazepine, Lamictal, Topamax, Neurontin, Sabril, and an excipient.

12. The oral drug tablet manufacturing method as claimed in claim 6, wherein the medicine powders at least comprise sildenafil, tadalafil, microcrystalline cellulose, croscarmellose sodium, nitroglycerin, and an excipient.

13. The oral drug tablet manufacturing method as claimed in claim 10, wherein the excipient can be microcrystalline cellulose, calcium phosphate polyvinylpyrrolidone, guar gum, sodium stearyl fumarate, xylitol, sodium gluconate, magnesium aluminate, maltose, mannitol, polyvinyl acetate, or sorbitol, or a combination of the above.

14. The oral drug tablet manufacturing method as claimed in claim 11, wherein the excipient can be microcrystalline cellulose, calcium phosphate polyvinylpyrrolidone, guar gum, sodium stearyl fumarate, xylitol, sodium gluconate, magnesium aluminate, maltose, mannitol, polyvinyl acetate, or sorbitol, or a combination of the above.

15. The oral drug tablet manufacturing method as claimed in claim 12, wherein the excipient can be microcrystalline cellulose, calcium phosphate polyvinylpyrrolidone, guar gum, sodium stearyl fumarate, xylitol, sodium gluconate, magnesium aluminate, maltose, mannitol, polyvinyl acetate, or sorbitol, or a combination of the above.
